(19) United States
(12) Patent Application Publication
(10) Pub. No.: US 2006/0257341 A1
(43) Pub. Date: Nov. 16, 2006

(54) Multicolor cosmetic product

(75) Inventors: Renato Ancorotti, Crema (IT); Luigi Gandini, Cavenago di Brianza (IT)

Correspondence Address: NIXON & VANDERHYE, PC, 901 NORTH GLEBE ROAD, 11TH FLOOR, ARLINGTON, VA, 22203, US

(73) Assignee: GAMMA CROMA S.P.A., Milan (IT)

(21) Appl. No.: 10/558,633

(22) PCT Filed: May 24, 2004
(86) PCT No.: PCT/IB04/01671

§ 371 Date: Jan. 17, 2006

(30) Foreign Application Priority Data
May 30, 2003 (IT) MI2003 A 001093

(51) Int. Cl. A61K 8/37

(52) U.S. Cl. 424/63

(57) ABSTRACT

The invention concerns a multicolor cosmetic product with solid consistence that comprises two or more cosmetic products of different colour mixed together in a predefined or random way, at least two of said cosmetic products of different colour being substantially present and distinguishable from one another throughout the thickness of said multicolor cosmetic product and a process for its preparation.

This invention concerns new multicolor cosmetic products suitable for make-up and a process for their preparation.

In particular, the invention concerns new multicolor cosmetic products having a solid consistence, an excellent powdery marking effect and the appearance of a variously coloured and variegated stone like marble.

### BACKGROUND OF THE INVENTION

The applicant filed an Italian patent application on 28th December 2001 application number M12001A002841, filed abroad as international application PCT/IB02/05257, claiming a multi-step process for the preparation of cosmetic products to obtain monochromatic cosmetic products for make-up featuring a solid consistence so that they can be produced in different formats and with the characteristic of being easy to apply while at the same time having a good powdery marking effect.

Face and body make-up cosmetics such as blusher and eye shadow are known. These products are normally produced in the form of loose or compacted powders. Make-up cosmetics are also known consisting of a combination of different cosmetic products in powder or poured, differently coloured, and arranged next to each other in supporting housing.

Multicolour products have also been prepared by mixing and pressing variously coloured powders.

### SUMMARY OF THE INVENTION

It has now been found that it is possible to produce solid multicolor cosmetics in which the different colours are variously mixed while remaining clearly distinguishable from one another in all the solid product this creating, according to the work process adopted, an effect that is variegated, veined, marble-like, spiraliform etc.

### DRAWINGS

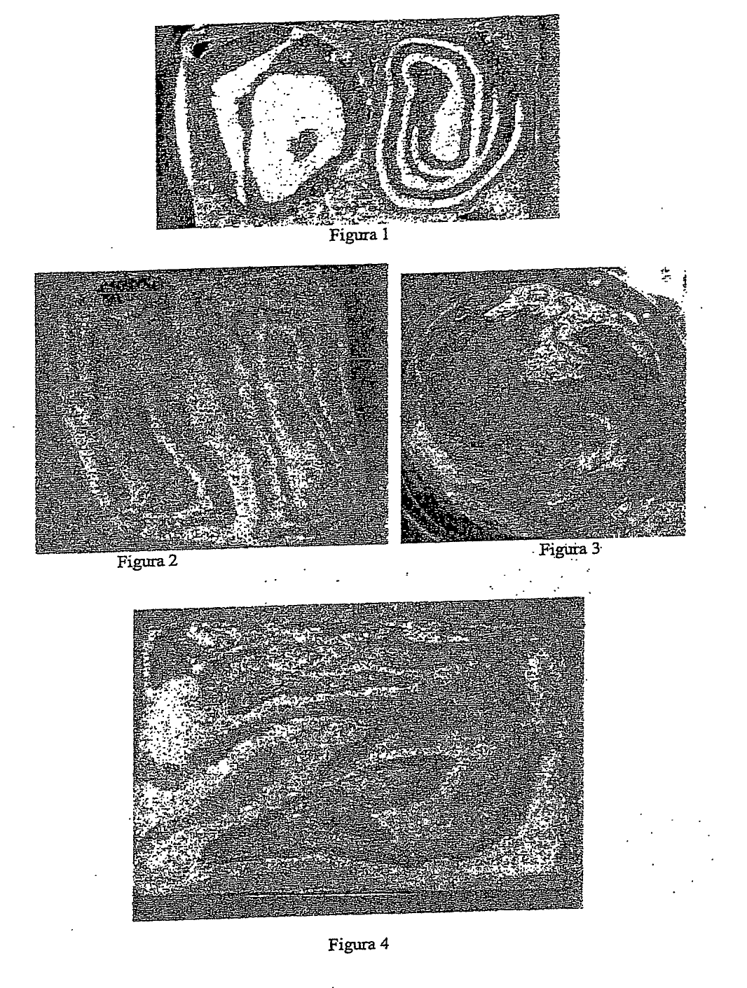

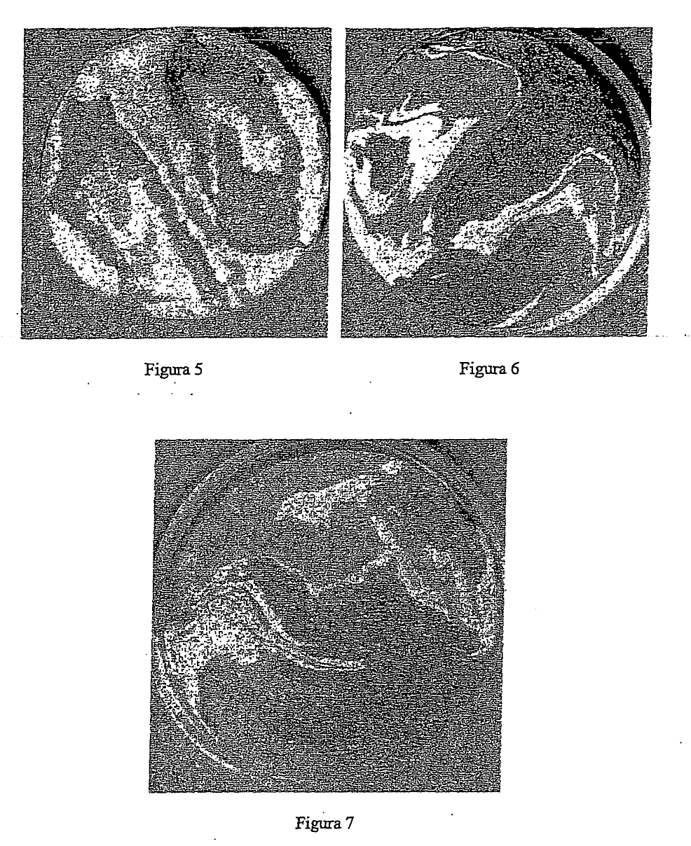

The figures from 1 to 7 show some examples of the multicolor result that can be obtained, but many other colour compositions, intentional or deriving from random mixing, can be obtained. In particular, FIGS. 1 and 2 show mixtures created by folding or rolling the variously coloured cosmetic products, while FIGS. 3 to 7 show random mixtures obtained with a mixer. FIGS. 1 to 7 are black and white reproductions of multicoloured products.

### DETAILED DESCRIPTION OF THE INVENTION

According to one of its aspects, the invention concerns a new multicolor cosmetic product with solid consistence characterised in that it comprises two or more cosmetic products of different colour mixed together in a predefined or random way, at least two of said cosmetic products of different colour being substantially present and distinguishable from each other throughout the thickness of said multicolor cosmetic product.

According to the present invention, “multicolor cosmetic product with solid consistence” indicates a cosmetic product suitable for make-up of the face and body with a consistence such that it can be processed, sized and packed as a solid, clearly different from the loose or compacted powders, and from poured products. In the “multicolor cosmetic product with solid consistence” of the invention the colours are clearly evident and separate from one another forming random or predefined variegations, thus having an appearance like multicoloured marble or camouflage fabric or featuring abstract designs with whorls, waves etc., said colours, or at least two of them, being present substantially in all the multicolor cosmetic product.

The multicolor cosmetic product with solid consistence of the invention can be obtained via a process that provides for the mixing of at least two viscous variously coloured cosmetic products, sizing and subsequent drying of the multicolor mixture obtained. Advantageously the coloured cosmetic products are extrusion products.

“Coloured products” here means products of any colour, including black and white, or pearly or glitter or otherwise decorative products, suitable for cosmetic use.

Variously coloured extrusion cosmetic products can be obtained with the process, as described in the Italian and international patent applications referred to above, which will become public at the time of publication of this patent application.

In short, the variously coloured cosmetic products can be obtained via a process that comprises the extrusion of a paste obtained by mixing of an emulsion of fats for cosmetic use with colouring powders. Once extruded and while still damp, the coloured cosmetic products are placed next to one another and processed in order to obtain the required chromatic effect; for example they can be folded or rolled into a spiral or placed in a mixer to obtain random and more thorough mixing of the different colours, thus obtaining a marble or a “camouflage” effect. Alternatively, the coloured cosmetic products can simply be laid one on the other to form strips of different colour. Once mixing has been completed, the multicolor cosmetic product, still damp, is sized as required and oven-dried.

The invention also concerns a process for the preparation of a multicolor cosmetic product with solid consistence that comprises preparation of the coloured cosmetic products according to the stages from (a) to (c):

a) prepare two phases called here “colouring powders” and “emulsion of fats”;

b) mix the above phases;

c) extrude the paste obtained;

combine as required at least two extrusion products of phase (c) of different colour, size and dry the damp multicolor cosmetic product obtained.

If required, the end product can be further cut, finished or sized.

The expression “emulsion of fats” as used here is a mixture that can be obtained by treatment of fats for cosmetic use with a neutral or coloured solvent, for example water or any solvent suitable for cosmetic use, including their mixtures; an essential characteristic of the solvent used is that it must be possible to eliminate it by drying after extrusion at temperatures that do not alter the end product, advantageously at temperatures not exceeding 50° C. Water, being readily available and inexpensive, is the preferred solvent for this invention.

According to this invention, the expression “fats for cosmetic use” indicates any fatty material suitable for the preparation of cosmetics such as the esters of fatty acids, triglycerides, waxes, fruit and seed oil derivatives and extracts etc.

Useful fats for cosmetic use are, for example, sorbitan stearate, isopropyl stearate, caprylic/capric triglycerides, dipentaerythrityl hexahydroxystearate/stearate/rosinate (sold under the trademark Cosmol 168AR), magnesium myristate and olive oil.

According to this invention, the expression “colouring powders” indicates any powder, or mixture of powders, containing colouring pigments suitable for cosmetic use.

Suitable colouring powders are, for example, those obtained by mixing synthetic and/or natural pigments, matte or pearly, with inert powders as diluents such as mica or talc, in varying quantities according to the powdery effect and colouring power required.

The colouring powders are generally prepared by mixing the required pigments with inert substances such as talc, silica and mica. According to a preferred aspect, parabenes and sodium dehydroacetate are added to the mixture which have the function of preserving the entire product during all the work phases.

The “fatty emulsion” and “colouring powders” phases are preferably mixed in equal quantities, i.e. 50-50% by weight.

The extrusion process of this invention can be performed by passing the paste (neutral or coloured) obtained in phase (b) through a normal extruder (or drawing machine) to obtain a semi-solid product, still damp, of the required shape.

The extruders useful in the process of the invention are, for example, piston or screw extruders. The product comes out of the extruder in different shapes according to the dies used (for example cylinder, parallelepiped etc.) and is cut to the required length.

The different viscous coloured cosmetic products can be mixed, as said, in a mixer, for example of the planetary type, but mixing can also be performed by hand or with appropriate machines to obtain only spiral patterns or simple overlapping of different coloured cosmetic products, as shown FIG. 1.

The mixing operation generally lasts from a few seconds to a few minutes, according to the type of chromatic effect required. Once the mixing has been completed, the multicolor cosmetic product still damp is cut into the required form and then dried.

Two or more coloured cosmetic products can be mixed, for example 3 or 4 or more, according to the chromatic effect required and the use for which the product is intended. For example coloured cosmetic products of similar colours can be mixed to obtain chromatic effects based on variations in tone of the same colour or coloured cosmetic products of clearly different shades or coloured cosmetic products can be mixed with matte or pearly pigments.

The drying phase of the process of the invention can be performed according to the conventional techniques, for example in an oven, in a vacuum or with fluidised bed on condition that said process creates conditions that ensure the evaporation of almost all the solvent used for preparation of the paste to be extruded, whatever it is. Obviously lower temperatures require longer drying times whereas at higher temperatures drying times will be shorter. The drying (or baking) will be preferably performed at low temperatures in order to permit slow elimination of the solvent from the paste and to obtain a well-compacted and uniform end product, without altering the end product.

Advantageously, the drying is performed in an oven at temperatures of 35-55° C., around 40° C. for example, until almost complete evaporation of the solvent, for example until the residual humidity is below or equal to 5%.

The machines and processing techniques individually used in the process of the invention are known to persons skilled in the art.

Other components, inert or non-inert, can be added to the colouring powder and fatty emulsion phases of the invention or directly to the paste to be extruded. As an example, additives can be added, as solid and liquid preserving agents, such as sodium benzoate, thickeners such as starch and its derivatives, diluents, gelling agents and adhesives, in order to make the paste obtained by mixture of the two phases suitable for extrusion, various inert powders, fragrances if required, etc. Said additives and their properties and uses in the field of cosmetics are well known to persons skilled in the art.

Once drying has been completed, the multicolor cosmetic product appears to all intents and purposes like a solid while at the same time facilitating pick-up with ordinary make-up tools (brushes, sponges etc.) for easy application on the area in question.

The process of the invention therefore permits the production of innovative multicolor cosmetics for decorative or curative make-up that represent a valid original alternative to the conventional products in powder, loose or compacted, and to the creamy type products. The products that can be obtained with the process of the invention represent another aspect of the invention.

Different multicolor cosmetic products of the invention can be combined in appropriate containers, for example side by side or assembled in one single container, to form original chromatic effects and/or to offer the user various make-up possibilities in one single solution.

The following examples are designed to illustrate the invention without limiting it in any way. It is understood that technically equivalent alternatives within reach of persons skilled in the art, even if not explicitly indicated, come under protection of the invention.

### EXAMPLE 1

General Operating Method

Appropriately clean and sanitise all the machinery required for the work process.

Preparation of the Fatty Emulsion

Weigh the water in an emulsifier; add the selected preservative and mix until fully dispersed. Add any thickeners and the mixture of liquid preservatives and disperse by means of a homogeniser. Weigh and dissolve the fatty mass (stearates, triglycerides) at approximately 60° C., add it to the previously prepared mixture and homogenise. Add some gelling agent and process for a few minutes. Check the viscosity and pour into clean drums.

Preparation of the Colouring Powders

Weigh the inert components, binders and any preservatives (talc, parabenes etc.) and grind for a few minutes. Weigh and add the raw materials making up the required colour and grind until obtaining a uniform mixture.

Preparation of the Taste to be Extruded

Mix the fatty emulsion and colouring powders for approximately 10 minutes in a mixer. Empty into clean bags and extrude in the drawing machine.

Mixtures of Two or More Coloured Cosmetics and Drying of the Multicolor Cosmetic Product Obtained

In a planetary type mixer, mix two or more extrusion products from the previous phase for a few seconds. Size and bake the multicolor product for 3 days at approximately 50° C.

Variation: place two or more extrusion products of the previous phase on top of one another and fold them or roll them to form spirals. Size and bake the multicolor product for 3 days at approximately 50° C.

### EXAMPLE 2

Quality-Quantity Composition of a Typical Paste Before Extrusion

| | |
|---|---|
| Water | 39.40% |
| Fats for cosmetic use (mixture of isopropyl stearate, | 6.00% |
| di-C12-13 alkyl malate, Cosmol 168AR) | |
| Pigments in a mixture with mica | 38.4% |
| Xanthan gum | 0.05% |
| Preservatives (mixture of sorbic acid and parabenes) | 1.1% |
| Inert components (mixture of zea mays, nylon | 14.55% |
| powder, talc) | |
| Fragrance | 0.5% |

1. Multicolor cosmetic product with solid consistence characterised in that it comprises two or more cosmetic products of different colour mixed together in a pre-defined or random way, at least two of said cosmetic products of different colour being substantially present and distinguishable from one another throughout the thickness of said multicolor cosmetic product.

2. Multicolor cosmetic product according to claim 1, characterised in that said cosmetic products of different colour are extruded products.

3. Multicolor cosmetic product according to claim 2, characterised in that said cosmetic products of different colour are placed side by side and mixed after extrusion, then sized and dried.

4. Multicolor cosmetic product according to claim 3, characterised in that said cosmetic products of different colour are mixed at random in a mixer.

5. Multicolor cosmetic product according to claim 3, characterised in that said cosmetic products of different colour are mixed together to form variously coloured layers, marbled effects, waves or spirals.

6. Multicolor cosmetic product according to claim 2 characterised in that said cosmetic products of different colour can be obtained by extrusion of a paste obtained by mixing of an emulsion of fats for cosmetic use with colouring powders.

7. Process for the preparation of a multicolor cosmetic product according to claim 1, having solid consistence which comprises preparation of the coloured cosmetic products according to the following phases from (a) to (c):
a) prepare two phases, one of “colouring powders” and one of “emulsion of fats”;
b) mix said phases;
c) extrude the paste obtained;
combine as required at least two extrusion products of phase (c) to obtain the required multicolor effect, size and dry the damp multicolor cosmetic product obtained.

8. Process according to claim 7, in which drying is performed at a temperature of 35-55° C.

9. Process according to claim 7, in which the dried multicolor cosmetic product is further processed in order to modify its dimensions and/or form.

10. Process according to claim 7, in which different multicolor cosmetic products are combined in appropriate containers.

11. Cosmetic product obtainable via the process of claim 7.

12. Cosmetic product of claim 1 which is an eyeshadow, a foundation or a blusher.

13. Use of the product of claim 1 as a cosmetic.
